(19) United States
(12) Patent Application Publication
(10) Pub. No.: US 2023/0068508 A1
(43) Pub. Date: Mar. 2, 2023

(54) HEALTHCARE SYSTEM WITH COMBINING TV VIEWING DATA AND BIO DATA

(71) Applicant: ATAM CO., LTD., Seoul (KR)

(72) Inventors: Sung Hee LEE, Seongnam-si, Gyeonggi-do (KR); Seung Baek LEE, Goyang-si, Gyeonggi-do (KR)

(73) Assignee: ATAM CO., LTD., Seoul (KR)

(21) Appl. No.: 17/799,942

(22) PCT Filed: Apr. 15, 2021
(86) PCT No.: PCT/KR2021/004766

§ 371 Date: Aug. 16, 2022

(30) Foreign Application Priority Data
Apr. 16, 2020 (KR) 10-2020-0046091

(51) Int. Cl. G16H 50/30; G16H 40/67; G16H 20/00; G08B 21/04

(52) U.S. Cl. CPC G16H 50/30; G16H 40/67; G16H 20/00; G08B 21/0423

(57) ABSTRACT

A healthcare system with combining TV viewing data and bio data includes a smart meter that collects and transmits first audio data of a first broadcast content, and collects and transmits ambient sounds; a smart device that collects and transmits second audio data of a second broadcast content, and collects and transmits usage data of an installed application; a wearable device that collects and transmits the user's bio data; a database storing broadcast reference data comparable to at least one of the first audio data and the second audio data; a broadcast content determining unit that compares one or more of the first audio data and the second audio data with the broadcast reference data to specify a third broadcast content; and an abnormality detection unit that determines that an abnormality has occurred in the user's personal safety.

### TECHNICAL FIELD

The present invention relates to a healthcare system, and more specifically, to a healthcare system with combining TV viewing data and bio data that determines whether an abnormality has occurred in the user's personal safety using the user's viewing record and the user's bio data to view the broadcast content, and notifies the user when an abnormality occurs.

### BACKGROUND SKILLS

One of the important issues of modern society is nuclear familyization and aging. In particular, the number of the elderly living alone, in which only the elderly live alone in the home, is gradually increasing. Since such elderly people living alone are in an environment where they cannot receive help in daily or emergency situations, they will be at great risk if a 365 days 24 hours management system is not established for the elderly living alone, and appropriate care services for the globally growing elderly population and single-person households are becoming a major social issue.

The following technologies have been introduced through such care services and management. The problem of the conventional technology, which requires the person in charge of the social welfare center to visit the home of the elderly living alone to check the current status of the elderly living alone, was improved through an automated system wherein it is possible to check the current status of the elderly living alone by calling the elderly living alone through the call button in the house and making frequent calls through the connection of the call center.

However, the conventional technology as described above simply relies on the automation system and only checks the current status of the elderly living alone on a one-time basis and writes documents by a social welfare officer or call center employee based on consultation with the elderly living alone through calls and phones.

In addition, although the health management system through IPTV has been introduced, there is a problem that it is not possible to accurately determine whether the TV is actually viewed because it is judged that TV is viewed according to the on/off of the power of the set-top box connected to the IPTV, regardless of the actual TV power on/off.

### DETAILED DESCRIPTION OF THE INVENTION

### Technical Challenges

The technical problem that the present invention intends to solve is to accurately measure the user's viewing record of viewing the broadcast content, and combine it with the user's bio data to determine whether an abnormality has occurred in the user's personal safety, and to provide a healthcare system that notifies if an abnormality occurs.

### Means of Solving a Problem

In order to solve the above technical problems, the healthcare system with combining TV viewing data and bio data according to an embodiment of the present invention is comprising: a smart meter that collects and transmits first audio data of a first broadcast content that a user is viewing through a first broadcast medium, and collects and transmits ambient sounds; a smart device that collects and transmits second audio data of a second broadcast content that the user is viewing through a second broadcast medium, and collects and transmits usage data of an installed application; a wearable device that collects and transmits the user's bio data; a database storing broadcast reference data comparable to at least one of the first audio data and the second audio data, storing at least one of the first audio data and the second audio data, storing the ambient sound as lifestyle data, storing usage data of the application, and storing the bio data; a broadcast content determining unit that compares one or more of the first audio data and the second audio data with the broadcast reference data to specify a third broadcast content that the user is viewing; and an abnormality detection unit that determines that an abnormality has occurred in the user's personal safety when the living pattern deviates from the user's existing living pattern by more than a certain range after analyzing the user's living pattern using the specified viewing data of the third broadcast content, the lifestyle data, the application usage data, and the bio data.

For example, the data measured above may be collectively referred to as user's living data.

The health care system in which TV viewing data and bio data are combined according to an embodiment of the present invention may further include an abnormality warning unit that notifies an abnormality warning when an abnormality occurs in the user's personal safety.

In one embodiment, the abnormal occurrence warning unit may classify the user as an intensive monitoring subject when an abnormality occurs first in user's personal safety, and notify an abnormality warning when an abnormality occurs again in the intensive monitoring subject's personal safety.

In one embodiment, when there are a plurality of users, it may be characterized in that a personal identification number for distinguishing each user is assigned.

In one embodiment, the bio data may include one or more of the user's heart rate, sleep time, exercise information, pulse, body temperature, and location.

### Effects of the Invention

As described above, since the present invention measures the viewing record based on the sound of the broadcast content that the user is actually viewing, it is possible to accurately measure the viewing record regardless of the power on/off state of the set-top box.

According to the present invention, when an abnormality occurs in the user's personal safety, by notifying it, the user can receive help quickly.

The present invention can continuously monitor whether an abnormality has occurred in the user's personal safety by creating a user's living pattern.

### BRIEF DESCRIPTION OF THE DRAWING

FIG. 1 is a conceptual drawing for explaining a healthcare system in which TV viewing data and bio data are combined according to an embodiment of the present invention.

FIG. 2 is a conceptual drawing for explaining a healthcare system in which TV viewing data and bio data are combined according to another embodiment of the present invention.

FIG. 3 is a drawing for explaining data collected for analyzing a user's living pattern.

FIG. 4 is a drawing for explaining the generated user's living pattern data.

FIG. 5 is a drawing for explaining a method of comparing the generated user's pattern data with the user's living data.

FIG. 6 is another drawing for explaining a method of comparing the generated user's pattern data with the user's living data.

### EMBODIMENT OF THE INVENTION

Since the present invention may have various changes and may have various forms, embodiments will be described in detail in the text. However, this is not intended to limit the present invention to the specific disclosed form, it should be understood to include all modifications, equivalents and substitutes included in the spirit and scope of the present invention. In describing each figure, similar reference numerals have been used for similar elements. Terms such as first, second, etc. may be used to describe various elements, but the elements should not be limited by the terms.

The above terms are used only for the purpose of distinguishing one element from another. The terms used in the present application are only used to describe specific embodiments, and are not intended to limit the present invention. The singular expression includes the plural expression unless the context clearly dictates otherwise.

In the present application, the term “including” or “composed” is to designate that a feature, a number, a step, an operation, an element, or a combination thereof described in the specification is present, and it should be understood that the presence or addition of one or more other features or a number, a step, an action, an element, a part, or a combination thereof is not excluded in advance.

Unless defined otherwise, all terms used herein, including technical or scientific terms, have the same meaning as commonly understood by one of ordinary skill in the art to which this invention belongs. Terms such as those defined in commonly used dictionaries should be interpreted as having a meaning consistent with the meaning in the context of the related art, and should not be interpreted in an ideal or excessively formal meaning unless explicitly defined in the present application.

Hereinafter, preferred embodiments of the present invention will be described in more detail with reference to the drawings.

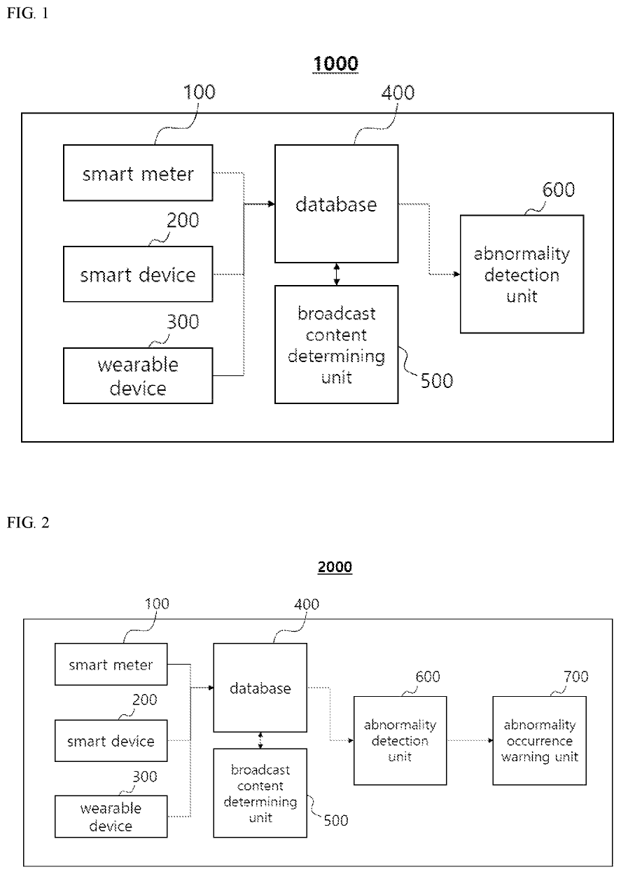

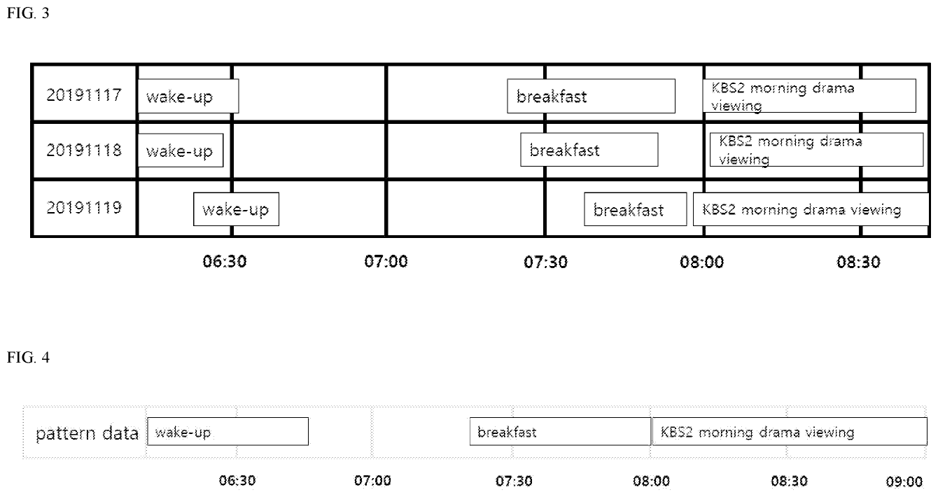

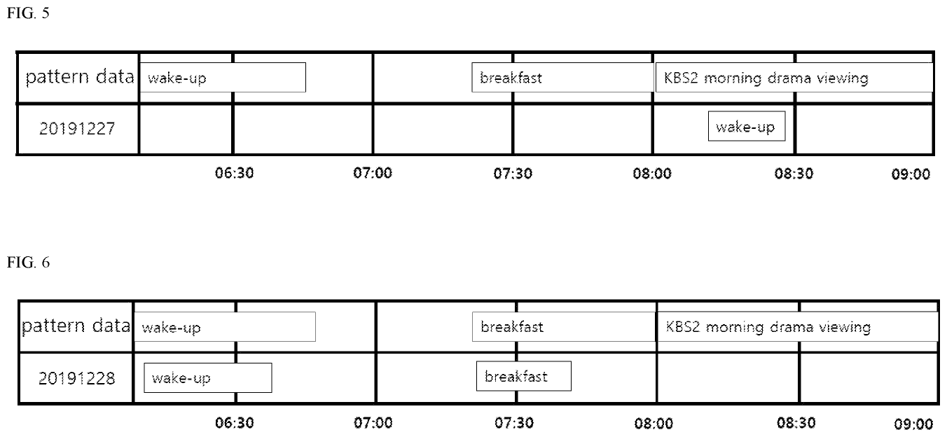

FIG. 1 is a conceptual drawing for explaining a healthcare system in which TV viewing data and bio data are combined according to an embodiment of the present invention, FIG. 2 is a conceptual drawing for explaining a healthcare system in which TV viewing data and bio data are combined according to another embodiment of the present invention, FIG. 3 is a drawing for explaining data collected for analyzing a user's living pattern, FIG. 4 is a drawing for explaining the generated user's living pattern data, FIG. 5 is a drawing for explaining a method of comparing the generated user's pattern data with the user's living data, and FIG. 6 is another drawing for explaining a method of comparing the generated user's pattern data with the user's living data.

Referring to FIG. 1, a healthcare system (1000) that combines TV viewing data and bio data according to an embodiment of the present invention includes a smart meter (100), a smart device (200), a wearable device (300), and a database (400), a broadcast content determining unit (500), and an abnormality detection unit (600).

The smart meter (100) may collect and transmit first audio data of the first broadcast content that the user is viewing through the first broadcast medium.

For example, the smart meter (100) may refer to an audience rating measuring device installed in a household, and a smart meter of ATAM Co., Ltd. may be used. The first broadcast medium is an electronic device that outputs broadcast content, and may mean, for example, a TV. The smart meter (100) can record sound, and has a built-in microphone for sound recording.

For example, the first broadcast content means broadcast content output through the first broadcast medium, and the first broadcast content may be a drama, news, movie, etc. broadcast through a TV.

For example, the first audio data means audio output through the TV when the first broadcast content is output, and the first audio data may be collected through the smart meter (100).

In addition, the smart meter (100) may collect and transmit ambient sounds. Ambient sound may mean within a certain distance range based on the place where the smart meter (100) is installed, for example, up to a distance up to a radius of 2M from the smart meter (100) may be defined as surrounding.

Ambient sounds may mean sounds such as conversations, phone calls, meals, and moving in home that occur during the user's living, but is not limited thereto.

The first audio data and ambient sound collected through the smart meter (100) may be converted into digital data and transmitted.

The smart device (200) may collect and transmit second audio data of the second broadcast content that the user is viewing through the second broadcast medium.

For example, the second broadcast medium may mean a smartphone, the second broadcast content may mean broadcast content output through the smartphone, and the second broadcast content may mean a drama, news, or movie, etc. output through the smartphone.

In addition, the smart device (200) may collect and transmit usage data of the installed application. The usage data may refer to various pieces of information that can inform the user's smartphone usage status, such as data on the start and end of applications, frequently used applications among a plurality of applications, the number of accesses, and access times.

In addition, the smart device (200) may also collect and transmit location information.

The wearable device (300) may collect and transmit the user's bio data. For example, the wearable device (300) may refer to a device that is attached to a user's body to measure and transmit the user's bio data, and a smart watch, a smart band, etc. may be used as the wearable device (300).

For example, the bio data may include one or more of a user's heart rate, sleep time, exercise information, pulse, body temperature, and location.

The database (400) may store broadcast reference data that can be compared with one or more of the first audio data and the second audio data. For example, the broadcast reference data may mean real-time or non-real-time broadcast content, and the broadcast content may include various programs such as dramas, movies, documentaries, and entertainment programs, etc.

The database (400) may store one or more of the first audio data and the second audio data, and may store application usage data and bio data.

Also, the database (400) may store ambient sounds as lifestyle data. For example, all data stored in the database (400) may be stored digitally.

For example, when there are a plurality of users, data may be stored in the database (400) by assigning each user a personal identification number capable of distinguishing each user.

The broadcast content determining unit (500) may compare one or more of the first audio data and the second audio data with the broadcast reference data to specify the third broadcast content that the user is viewing.

For example, the third broadcast content may mean broadcast content that the user is viewing through the first broadcast medium or the second broadcast medium.

For example, by comparing the audio fingerprinting data of the first audio data and the broadcast reference data with each other, it is possible to specify which broadcast content the first broadcast content currently being viewed by the user corresponds to.

As another example, a method of specifying which broadcast content corresponds to the first broadcast content currently viewed by the user may be used by comparing the audio data of the first audio data and the broadcast reference data with each other.

Since the method of specifying the third broadcast content by comparing the second audio data with the broadcast reference data is the same as described above, a detailed description thereof will be omitted.

When the third broadcast content is specified, the user's viewing record may be additionally stored in the database (400). The viewing record may refer to various data related to viewing, such as viewing start time, viewing end time, and viewing frequency.

In the case of conventional IPTV and digital cable TV, since if the TV power is off, but the TV broadcast receiver, set-top box, is turned on, the TV broadcast reception status is continuously maintained, even when the user does not actually view the broadcast content, the viewing record may be stored as viewing the broadcast content.

In other words, even if there is a problem that accurate viewing records cannot be reflected when the viewing records are stored based on the set-top box, since the healthcare system (1000) that combines TV viewing data and bio data according to an embodiment of the present invention compares the audio of the broadcast content viewed by the user to measure the broadcast content viewed by the user, it has the advantage of being able to store accurate viewing records (viewing data) of broadcast content.

The abnormality detection unit (600) may analyze the user's living pattern using viewing data, lifestyle data, application usage data, and bio data of the specified third broadcast content.

For example, viewing data, lifestyle data, application usage data, and bio data stored in the database (400) may be pre-processed into learning data suitable for an artificial intelligence (AI) analysis algorithm.

A user's living pattern may be generated by analyzing data collected over a long period of time with an artificial intelligence (AI) analysis algorithm.

Referring to FIG. 3, the wake-up time, breakfast time, and TV viewing record by date for three days are shown. The abnormality detection unit 600 may analyze a living pattern as shown in FIG. 4 based on data for three days.

For example, in the case of waking up time, since there is a certain time difference based on 6:30 am, a living pattern of waking up at about 6:00 am to 6:45 am may be created. In addition, pattern data may be generated by analyzing living patterns by applying the same method to breakfast and drama viewing time.

When the analyzed living pattern deviates from the user's existing living pattern by more than a certain range, the abnormality detection unit (600) may determine that an abnormality has occurred in the user's personal safety.

Referring to FIG. 5, 2019 Dec. 27. Since it appears that the user's wake-up time is after 8 am, this is a significant difference from the existing generated living pattern, and thus it can be determined that an abnormality has occurred in the user's personal safety.

Referring to FIG. 6, 2019 Dec. 28. Both the user's wake-up time and breakfast time fall within the range of the generated living pattern, but since it appears that there is no morning drama viewing, it can be determined that an abnormality has occurred in the user's personal safety.

For example, if the user's living data is different from the generated living pattern or greatly deviates from the living pattern, the user can be informed that the living pattern is deviated through at least one of the smart device (200) and the wearable device (300). Through this, the user can recognize that his/her behavior is different from the existing living pattern, thereby raising the user's awareness in advance.

The health care system (2000) that combines TV viewing data and bio data according to another embodiment of the present invention includes a smart meter (100), a smart device (200), a wearable device (300), a database (400), a broadcast content determining unit (500), an abnormality detection unit (600), and an abnormality occurrence warning unit (700).

Since the smart meter (100), the smart device (200), the wearable device (300), the database (400), the broadcast content determining unit (500), and the abnormality detection unit (600) are the same as described above, detailed descriptions will be omitted.

The abnormality occurrence warning unit (700) may notify an abnormality warning when an abnormality occurs in the user's personal safety.

For example, when it is determined that an abnormality has occurred in the user's personal safety, the abnormality occurrence warning unit (700) may notify a person registered in advance, such as a smartphone of a family member, the government, a local government, and a hospital.

As an example, the abnormality occurrence warning unit (700) may notify an abnormality warning through a message or a messenger, but there is no limitation in the method.

The abnormality occurrence warning unit (700) may classify the user as a subject for intensive monitoring when an abnormality occurs in the user's personal safety for the first time, and notify an abnormal warning when abnormality occurs again in the personal safety of the subject of intensive monitoring.

For example, even if the user's wake-up time deviates from the existing living pattern data, an abnormal warning may not be notified if the user's living data after that does not deviate from the existing living pattern data. This is to reflect the case in which the user's living pattern can deviate at least once to prevent the reliability of the occurrence of an abnormal warning from being lowered. In addition, when the user's living data is again within the range of the existing living pattern data, the user is excluded from the subject of intensive monitoring.

However, even after it is first determined that an abnormality has occurred in the user's personal safety, if the user's living data is out of the range of the existing living pattern, an abnormality warning is provided so that the user's personal safety can be quickly protected.

In addition, according to the importance of the living data, even if it is the first time to deviate from the life pattern, an abnormality warning can be provided immediately. For example, if the user is a patient with asthma and wheezing is a typical symptom, but wheezing is collected, the abnormality occurrence warning unit (700) immediately notifies an abnormality warning so that the user can receive help quickly.

Although the embodiments according to the present invention have been described above, these are merely exemplary, and those of ordinary skill in the art will understand that various modifications and equivalent ranges of embodiments are possible therefrom.

1. A healthcare system with combining TV viewing data and bio data comprising:
a smart meter that collects and transmits first audio data of a first broadcast content that a user is viewing through a first broadcast medium, and collects and transmits ambient sounds;
a smart device that collects and transmits second audio data of a second broadcast content that the user is viewing through a second broadcast medium, and collects and transmits usage data of an installed application;
a wearable device that collects and transmits the user's bio data;
a database storing broadcast reference data comparable to at least one of the first audio data and the second audio data, storing at least one of the first audio data and the second audio data, storing the ambient sound as lifestyle data, storing usage data of the application, and storing the bio data;
a broadcast content determining unit that compares one or more of the first audio data and the second audio data with the broadcast reference data to specify a third broadcast content that the user is viewing; and
an abnormality detection unit that determines that an abnormality has occurred in the user's personal safety when the living pattern deviates from the user's existing living pattern by more than a certain range after analyzing the user's living pattern using the specified viewing data of the third broadcast content, the lifestyle data, the application usage data, and the bio data.

2. The healthcare system of claim 1, wherein the healthcare system further comprises an abnormality occurrence warning unit notifying an abnormality warning when an abnormality occurs in the user's personal safety.

3. The healthcare system of claim 2, wherein the abnormality occurrence warning unit classifies the user as a subject for intensive monitoring when an abnormality occurs in the user's personal safety for the first time and provides an abnormality warning when an abnormality occurs again in the personal safety of the subject of intensive monitoring.

4. The healthcare system of claim 1, wherein the healthcare system is characterized in that if the user is plural, a personal identification number capable of distinguishing each user is assigned.

5. The healthcare system of claim 1, wherein the bio data comprises one or more of the user's heart rate, sleep time, exercise information, pulse, body temperature, and location.
